(19) United States
(12) Patent Application Publication
(10) Pub. No.: US 2012/0213766 A1
(43) Pub. Date: Aug. 23, 2012

(54) Anticancer Compounds from Vernonia Guineensis

(76) Inventors: Ngeh Joseph Toyang, Columbia, MD (US); Aloysius Nkwain Toyang, Fundong (CM); Theresia Ngoin Toyeng, Bamenda Upstation (CM)

(21) Appl. No.: 13/032,522

(22) Filed: Feb. 22, 2011

(51) Int. Cl. A61K 39/395; A61K 31/121; A61K 38/43; A61K 31/335; A61P 35/00; A61P 9/00; C07C 49/258; C07D 313/00

(52) U.S. Cl. 424/130.1; 568/413; 514/675; 549/346; 514/450; 424/94.1

(57) ABSTRACT

This invention relates to novel compounds with anticancer activity isolated from Vernonia guineensis (Asteraceae) having chemotherapeutic activity against abnormal cell growth such as cancers. The invention also relates to pharmaceutical compositions containing the compounds and to methods of treating abnormal cell growth such as cancer, including combination therapies thereof.

### FIELD OF THE INVENTION

This invention relates to novel compounds, compositions, methods of use of the novel compounds isolated from Vernonia guineensis Benth. (Asteraceae). These compounds possess anti-proliferative activity against abnormal cell growth such as cancer cells, particularly, breast cancer, colon cancer, Kaposi sarcoma, leukaemia, lung cancer, melanoma, ovarian cancer and prostate cancer. The compounds isolated from Vernonia guineensis have therapeutic efficacy alone or in combination with other pharmaceutical compositions intended for the prevention or treatment of cancers. The compounds of the invention can be prepared into pharmaceutically acceptable salts, prodrugs, tautomers or stereoisomers thereof.

### BACKGROUND OF THE INVENTION

Vernonia guineensis Benth. (Asteraceae) is a widely used medicinal plant in West Africa, particularly in Cameroon. Whole carrot-like tubers and powders derived therefrom; packaged in 10-20 gram sachets, are sold by herbalists in the open market. Claimed medicinal uses include adaptogenic properties to combat stress and as a stimulant. The plant is also used as an anthelmintic, an aphrodisiac, an antidote to poison, and to treat malaria and jaundice (Iwu, 1993). Fulani pastoralists in the North West Region of Cameroon feed the root powder, mixed with milk to promote growth and prevent helminth infestation in calves.

V. guineensis Benth. is a variable species with three varieties (var. guineensis, var. cameroonica, and var. procera) recognized in the West African region. They are herbaceous with strong erect stems from a perennial woody rootstock, 1.70 m high, distributed across the region from Mali to Western Cameroons, and across central Africa from Cameroon to Sudan (Burkill 1985). The genus Vernonia (Asteraceae) has more than 500 species. Many of the species are used as food and medicine in many parts of the tropics. Vernonia amygdalina commonly referred to as bitter leaf is one of the most popular greenleaf vegetables in Cameroon and other West African countries. Vernonia amygdalina is used in folk medicine to treat malaria, as an analgesic (Njan A A, Adzu B; Agaba A G, Byrugaba D; Diaz-Llera S, Bangsberg D R, J Medical foods, 11(3) 574-81, 2008, antiulcer, constipation, high blood pressure, and as a tonic (Iwalokun B A; Efedede B U; ALabi-Sofunde J A; Oduala T.; Magbagbeola O A; Akindwande A I, J. Med Food, 9(4) 524-30, 2006; in ethnoveterinary medicine as an anthelmintic (Toyang N. J., Nuwanyakpa M, Django S, Ndi C., Wirmum C. K., Indigenous Knowledae and Development Monitor, 1995, 3:20-22); and in zoopharmacognosy V. amygdalina is used by chimpanzees in self medication practices (Huffman, M. A., Seifu, M., 1989. Primates 30: 51-63). Other Vernonia species frequently used include Vernonia condensata, Vernonia cineria, vernonia galamensis and Vernonia colorata.

Early isolation work on V. guineensis Benth. was carried out by Toubiana et al (1975) who isolated two compounds called vernodalin and vernolapin. Tchinda et al. (2002 and 2003) reported the isolation of stigmatane derivatives and sucrose ester type compounds from V. guineensis Benth. with one of the stigmatanes showing anti-trypanocidal activity. Several bitter and non-bitter stigmastane-type steroids, the vernoniosides (Jisaka et al., 1992, 1993; Ponglux et al., 1992; Igile et al., 1995; Sanogo et al., 1998) have been isolated and characterized from the genus.

No prior report was found on the isolation of anticancer compounds from Vernonia guineensis. Noumi, 2010 documented the use of V. guineensis for the treatment of prostatic disease by traditional healers in Foumban, Cameroon. However, anticancer and cytotoxic activity has been reported for some members of the Vernonia genus including Vernonia amygdalina, Vernonia cinerea, Vernonia bockiana, Vernonia scorpioides, Vernonia chinensis, Vernonia pachyclada, Vernonia lasiopus, Vernonia hymenolepis. A search of the current scientific literature indicate over one hundred and twenty publications on the biological and chemical properties of the Vernonia genus out of which at least nineteen research articles are on the anticancer/cytotoxicity effects of members of the Vernonia genus. Eight of the nineteen publications on the anticancer/cytotoxicty of the genus Vernonia pattern to Vernonia amygdalina; Oyugi D A, Luo X, Lee K S, Hill B, Izeybigie E B, Exp Biol Med (Maywood) Jan. 28, 2009; Gresham L J, Ross J, Izevbigie E B, Int J Environ Res Public Health, 2008 5(5) 342-8; Yedjou C, Izevbigie E, Tchounwou P, Int J Environ Res Public Health, 2008 5(5) 337-41; Opata M M, Izevbigie E B, Int J Environ Res Public Health, 2006 3(2) 174-9; Howard C B, Stevens J, Izevbigie E B, Walker A, McDaniel O, Cell Mol Biol, 2003 49(7) 1057-65; Izevbigie E B, Exp Biol Med 2003 228(3) 293-8; Jisaka M, Ohigashi H, Takegawa K, Huffman M A, Koshmizu K, Biosci Biotechnol Biochem. 1993 57(5) 833-4; Kupchan S M, Hemingway R J, Karim A, Werner D. J Org. Chem. 1969 34(12) 3908-11.

The anticancer/cytotoxic compounds isolated from the Vernonias are mainly sesquiterpenes, Huo J, Yang S P, Xie B J, Liao S G, Lin L P, Ding J, Yue J M, J Asian Nat Prod Res. 2008 10(5-6) 571-5; Pagno T, Blind L Z Biavatti M W, Kreuger M R. Braz J Med. Res. 2006 39(11) 1483-91; Chen X, Zhan Z J, Zhang X W, Ding J, Yue J M. Planta Med. 2005 71(10) 949-54; Williams R B, Norris A, Siebodnick C, Merola J, Miller J S, Andriantsiferana R, Rasamison V E, Kingston D G. J Nat. Prod. 2005 68(9) 1371-4; Kuo Y H, Kuo Y J, Yu A S, Wu M D, Ong C W, Yang Kuo L M, Huang J T, Chen C F, Li S Y. Chem Pharm Bull. 2003 51(4) 425-6 amongst others. Sesquiterpenes especially those containing the lactone group (cyclic ester) have demonstrated significant antitumor activity and some of the promising ones are currently undergoing clinical trials including Parthenolides, Artemisinin derivatives and Thapsigargin (Ghantous A, Gali-Muhtasib H, Vuorela H, Saliba N A, Darwiche N. Drug Discovery Today 2010 15(16) 668-678). The Sesquiterpene lactones have become interesting candidates for cancer chemotherapy due to the fact that they seem to exhibit a unique mechanism of action by targeting cancer stem cells (Kawasaki B T, Prostate 2009 69:827-837).

Given that well over six million people a year die globally from cancer, coupled with the increasing rate of failure of current chemotherapies, there is a need to discover and develop new anticancer compounds. The compounds of the present invention are novel molecules that have demonstrated antitumor activity in cell lines and cancer models tested both in vitro and in vivo.

### SUMMARY OF THE INVENTION

Bioactivity guided fractionation of the extract of the leaf matter of Vernonia guineensis Benth., led to the isolation and identification of the anticancer molecules in this plant. Accordingly, the present invention provides anti-cancer agents comprising a range of novel derivatives based on compounds of the generic Formula I and Formula II.

Compounds of Formula I are represented by the general Formula I:

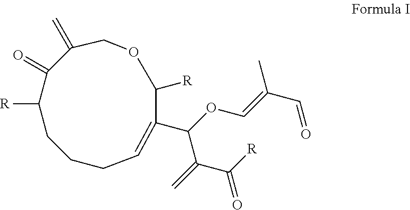

R is independently selected from (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy. In a preferred embodiment, “R” is a methyl (—CH₃), hydroxyl (—OH), methoxy (—OCH₃), ester (—OCH₂CH₃, —OCCH₂CH₂COOH). In a most preferred embodiment, “R” is a hydroxyl group, shown in Compound A (Kuminol) namely, 2-[(2,8-Dihydroxy-10-methylene-9-oxo-oxacycloundec-3-en-3-yl)-(2-methyl-3-oxo-propenyloxy)-methyl]-acrylic acid with a molecular formula of C₁₉H₂₄O₈ and molecular weight of 380.39.

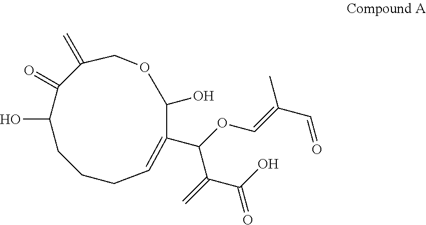

In another embodiment, anti-cancer compounds of the present invention comprise compounds derived from generic Formula II. X and Y are independently selected from (C₁-C₆)alkylC(O), (C₁-C₆)alkyl, (C₃-C₈)cyclo alkyl, (C₆-C₁₀) aryl, (C₁-C₁₀)hetero aryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, In a preferred embodiment, “X” and “Y” are a methyl (CH₃), hydroxyl (—OH), Methoxy (—OCH₃), ester (—OCH₂CH₃, —OCCH₂CH₂COOH).

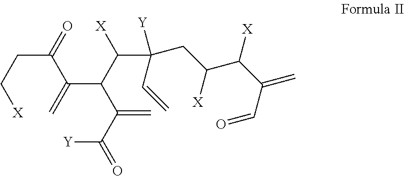

In a most preferred embodiment, “X” is hydroxyl and “Y” is a methyl as shown in Compound B (Vernoginin), namely 3,4,7,12,-Tetrahydroxy-6-methyl-2,9-dimethylene-8-(1-methylene-2-oxo-propyl)-10-oxo-6-vinyl-dodecanal with molecular formula of C₂₁H₃₀O₇ and molecular weight of 394.46.

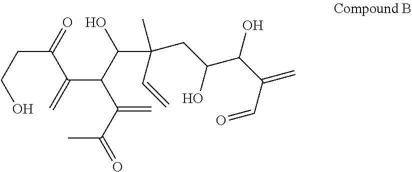

The compounds A and B are isolated from the extract of Vernonia guineensis Benth. The extraction is carried out by use of aqueous or organic solvents. The organic solvents used include alcohols such as methanol and ethanol, alkenes such as hexane, esters such as ethyl acetate, halogenated solvents such as chloromethane, chloroethane, ketones such as acetone. Ethyl acetate and acetone are highly preferred for the complete extraction of the anticancer compounds.

The compounds of the present invention can be applied to inhibit the growth of cancer cells in vivo and can also have greater than additive effect in combination with other chemotherapeutic compounds for the treatment of cancer. The compounds of the present invention can be applied to treat several cancers including breast cancer, colon cancer, Kaposi sarcoma, leukaemia, lung cancer, melanoma, ovarian cancer, prostate cancer, non-Hodgkin's lymphoma amongst other cancers.

It is another object of the present invention to provide a pharmaceutical composition comprising an effective amount for treating cancer of an agent selected from: a compound of the Formula I, wherein R is independently (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula I; or a prodrug or pharmaceutically active metabolite of a compound of the Formula I, or a pharmaceutically acceptable salt of a prodrug or metabolite thereof.

It is yet another object of the present invention to provide a pharmaceutical composition comprising an effective amount for treating cancer of an agent selected from: a compound of the Formula II, wherein X and Y are methyl, or hydroxyl, or selected from a substituted or unsubstituted alkyl, alkyloxy, aryl, aryloxy, heteroaryl, carbocycle, or heterocycle group, or a pharmaceutically acceptable salt of a compound of the Formula II, solvate or hydrate thereof; or a prodrug or pharmaceutically active metabolite of a compound of the Formula II, or a pharmaceutically acceptable salt of a prodrug or metabolite thereof.

It is another object of the present invention to provide pharmaceutically effective amount of pharmaceutical compositions for the treatment of cancers comprising compounds of the generic Formulas I and II, their pharmaceutically acceptable salts or prodrugs, either alone or in combination with other anticancer agents in proportions determined to be effective in inhibiting cancer cell proliferation and malignancies. Suitable pharmaceutical excipients include, but are not limited to alcohols, water, diluents, absorption enhancers, binders such as cellulose derivatives, disintegrants, fillers such as starch, colorants, coating agents, sweeteners and preservatives. The pharmaceutical composition of the present invention can be produced by mixing the compounds of the present invention alone or in combination with other anticancer agents in various pharmaceutical forms including tablets, powders, capsules, granules, syrups, sprays, injectables, suppositories and other pharmaceutically acceptable formulations.

Pharmaceutically acceptable salts include any salt which is capable of providing directly or indirectly a compound as described herein upon administration to the patient. Non-pharmaceutically acceptable salts also fall within the scope of this invention as they may be useful in the formulation of the pharmaceutically acceptable salts. Pharmaceutically acceptable salts can be prepared following established methods known by those skilled in the art.

It is another object of the invention to provide a method of treating cancer comprising administering to a patient in need of such treatment, a pharmaceutical composition comprising an effective amount for treating cancer selected from: a compound of the Formula I, wherein R is independently methyl, or hydroxyl, or (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula I; solvate or hydrate thereof, or a prodrug or pharmaceutically active metabolite of a compound of the Formula I, or a pharmaceutically acceptable salt of a prodrug or metabolite thereof.

It is yet another object of the invention to provide a method of treating cancer comprising administering to a patient in need of such treatment, a pharmaceutical composition comprising an effective amount for treating cancer selected from: a compound of the Formula II, wherein X and Y are independently methyl, or hydroxyl, or (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula II, solvate or hydrate thereof, or a prodrug or pharmaceutically active metabolite of a compound of the Formula I, or a pharmaceutically acceptable salt of a prodrug or metabolite thereof.

The compounds of the present invention may be used alone or in combination with and at least one anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

The compounds of the present invention may also be used alone or in combination with an amount of one or more substances selected from anti-angiogenesis agents, signal transduction inhibitors, and antiproliferative agents, which amounts are together effective in treating abnormal cell growth and hyperproliferative disorders.

An embodiment of the present invention includes those pharmaceutical compositions for the treatment of abnormal growth wherein the abnormal cell growth is cancer.

Other embodiments of the present invention include those pharmaceutical compositions for the treatment of abnormal cell growth, wherein the cell growth is a non-cancerous hyperproliferative disorder, such as benign hyperplasia of the skin or prostate

The present invention also relates to a pharmaceutical composition for the treatment of a disease relating to vasculogenesis or angiogenesis in a mammal comprising an amount of a compound of Formula I or II or a pharmaceutically acceptable salt, prodrug, solvate or hydrate thereof that is effective in treating said disease, and a pharmaceutically acceptable carrier. Examples of said disease include tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The present invention also relates to a method for the treatment of abnormal cell growth in a mammal comprising administering to said mammal an amount of a compound of Formula I or II or a pharmaceutically acceptable salt, prodrug, solvate or hydrate thereof that is effective in treating said abnormal cell growth. Examples of abnormal cell growth include cancer and non-cancerous cell growths such as benign hyperplasia of the skin or prostate.

An embodiment of the present invention includes those methods of treatment wherein the treatment is for vasculogenesis or angiogenesis.

An embodiment of the present invention includes those methods of treatment wherein the treatment is for angiogenesis.

The present invention also relates to a method for the treatment of a hyperproliferative disorder in a mammal which comprises administering to said mammal a therapeutically effective amount of a compound of formula I or II or a pharmaceutically acceptable salt, prodrug, solvate or hydrate thereof in combination with at least one anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

The present invention also relates to a method for the treatment of a hyperproliferative disorder in a mammal which comprises administering to said mammal an amount of a compound of formula I or a pharmaceutically acceptable salt, prodrug, solvate or hydrate thereof in combination with an amount of one or more substances selected from anti-angiogenesis agents, signal transduction inhibitors, and antiproliferative agents, which amounts are together effective in treating said hyperproliferative disorder.

The following illustrations and examples are used to further explain the embodiment in the present invention. The examples below should not, however, be considered to limit the scope of the invention as modifications will readily occur particularly when carried out by those skilled in the art, which modifications will be within the spirit of the invention and the scope of the claims herein appended.

### BRIEF DESCRIPTION OF THE DRAWINGS

FIG. 1: In-vitro antiproliferation assay of Kuminol and Vernoginin against non-Hodgkin's lymphoma cells.

FIG. 2. In-vitro antiproliferation assay of Kuminol and Vernoginin against Kaposi sarcoma cells.

FIG. 3. Annexin V detection of apoptosis effects of Kuminol (VIBO 002) and Vernoginin (NTVGL 002)

FIG. 4. Apoptotic effect of Kuminol (VIBO) and Vernoginin (NTVGL) on PC-3 cells after 24 h incubation.

FIG. 5. In vivo studies showing the anti-cancer effects of compounds of the present invention

### DETAILED DESCRIPTION OF THE INVENTION

The leaves of Vernonia guineensis were harvested from young plants and pulverized into powder after drying under shade. The ground material was extracted by use of aqueous or organic solvent. The organic solvents used include alcohols such as methanol and ethanol, alkenes such as Hexane, esters such as ethyl acetate, halogenated solvents such as chloromethane, chloroethane, ketones such as acetone. Ethyl acetate and acetone are highly preferred for the complete extraction of the anticancer compounds.

The extracts obtained as described above were fractionated by absorbing onto celite and then extracting with increasing polarities of ethyl acetate in hexane. Each fraction obtained was evaporated in-vacuo to remove the solvent and monitored by thin layer chromatography (TLC). Fractions that showed similarities were pooled. The dried residue of each fraction was used in the anticancer assay to determine effect on cell proliferation. The active fraction was subjected to dry column chromatography (DCC) using normal phase silica gel activated for visualization with UV 254. This procedure led to the isolation of compounds of Formula I in pure form. The Compound A (Kuminol) and the other fractions obtained were further subjected to anticancer activity screening.

The Compound A (Kuminol) and one of the fractions showing a different TLC profiles exhibited growth inhibition against prostate cancer, human melanoma, breast cancer and others. Further purification of second active fraction by use of Waters Sep Pak flash chromatography yielded compounds of the Formula II. Compound B (Vernoginin) inhibited prostate cancer, human melanoma, breast cancer and others in the in vitro cell proliferation assay.

The representative compounds of Formula I and II, namely Compounds A and B demonstrated anticancer properties in anticancer assays. The anticancer screen is based on the highly sensitive 3-(4,5-dimethylthiazol-2-yl)-2, S-diphenyl tetrazolium bromide (MTT) assay. Cell proliferation using cell lines of prostate cancer, breast cancer, human melanoma, lung cancer, ovarian cancer, leukaemia, colon cancer and more were screened. Cell proliferations in the above assays were hindered by the compounds A and B with very low half-maximal inhibitory concentrations (IC₅₀) values. Given the demonstrated anticancer activity in the cell proliferation assay, the compounds A and B can be used in inhibiting the cell growth of cancers including prostate cancer, breast cancer, human melanoma, lung cancer, ovarian cancer, leukaemia, colon cancer and more. This also implies that the compounds of Formula I and II can be applied in the treatment of cancers including prostate cancer, breast cancer, human melanoma, lung cancer, ovarian cancer, leukaemia, colon cancer, Kaposi sarcoma, non-Hodgkin's Lymphoma and more. The following examples further describe the details of the invention:

The compound of the invention can also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, and inhibitors of the receptor tyrosine kinase

Other anti-angiogenesis agents, including, but not limited to COX-II (cyclooxygenase II) inhibitors, other MMP (matrix-metalloproteinase) inhibitors, such as MMP-2 and MMP-9 inhibitors, other anti-VEGF antibodies or inhibitors of other effectors of vascularization can also be used in conjunction with the compound of formula I in the present invention.

Examples of useful COX-II inhibitors include CELEBREX™ (celecoxib), Bextra (valdecoxib), paracoxib, Vioxx (rofecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety.

Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The compounds of the present invention may be used alone or in combination with one or more of a variety of anti-cancer agents or supportive care agents. For example, the compounds of the present invention may be used with cytotoxic agents, e.g., one or more selected from the group consisting of a camptothecin, irinotecan HCl (Camptosar), edotecarin, SU-11248, epirubicin (Ellence), docetaxel (Taxotere), paclitaxel, rituximab (Rituxan) bevacizumab (Avastin), imatinib mesylate (Gleevac), Erbitux, gefitinib (Iressa), and combinations thereof. The invention also contemplates the use of the compounds of the present invention together with hormonal therapy, e.g., exemestane (Aroma-sin), Lupron, anastrozole (Arimidex), tamoxifen citrate (Nolvadex), Trelstar, and combinations thereof. Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds of the invention.

Alkylating agents include, but are not limited to: AMD-473, altretamine, AP-5280, apaziquone, brostallicin, bendamustine, busulfan, carboquone, carmustine, cyclophosphamide, estramustine, fotemustine, glufosfamide, ifosfamide, KW-2170, mafosfamide, melphalan, mitobronitol, mitolactol, nimustine, nitrogen mustard N-oxide, temozolomide, thiotepa and ranimustine. Platinum-coordinated alkylating compounds include but are not limited to, cisplatin, carboplatin, eptaplatin, lobaplatin, nedaplatin, oxaliplatin or satrplatin.

Antimetabolites include but are not limited to: 5-azacitidine, capecitabine, carmofur, cladribine, clofarabine, cytarabine, decitabine, doxifluridine, eflornithine, enocitabine, ethynylcytidine, 5-fluorouracil (5-FU) alone or in combination with leucovorin, leucovorin, cytosine arabinoside, hydroxyurea, fludarabine, TS-1, gemcitabine, methotrexate, melphalan, 6-mercaptopurine, mercaptopurine, nelarabine, nolatrexed, ocfosfate, disodium premetrexed, pentostatin, pelitrexol, raltitrexed, riboside, tegafur, triapine, trimetrexate, UFT, vidarabine, vincristine, vinorelbine,; or for example, one of the preferred anti-metabolites disclosed in European Patent Application No. 239362 such as N-(5-[N-(3,4-dihydro-2-methyl-4-oxoquinazolin-6-ylmethyl)-Nmethylamino]-2-thenoyl)-L-glutamic acid.

Antibiotics include but are not limited to: aclarubicin, actinomycin D, amrubicin, annamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin or zinostatin.

Hormonal therapy agents, e.g., exemestane (Aromasin), Lupron, anastrozole (Arimidex), doxercalciferol, fadrozole, formestane, anti-estrogens such as tamoxifen citrate (Nolvadex) and fulvestrant, Trelstar, toremifene, raloxifene, lasofoxifene, letrozole (Femara), or anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide) and combinations thereof.

Plant derived anti-tumor substances include for example those selected from mitotic inhibitors, for example vinblastine, vincristine, docetaxel (Taxotere) and paclitaxel.

Cytotoxic topoisomerase inhibiting agents include one or more agents selected from the group consisting of aclarubicn, amonafide, belotecan, camptothecin, 10-hy [symbol] droxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, and topotecan, and combinations thereof.

Immunologicals include interferons and numerous other immune enhancing agents. Interferons include interferon alpha, interferon alpha-2a, interferon, alpha-2b, interferon beta, interferon gamma-1a or interferon gamma-n1. Other agents include filgrastim, lentinan, sizofilan, TheraCys, ubenimex, WF-10, aldesleukin, alemtuzumab, BAM002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab, Provenge.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, or ubenimex.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, TLK-286 or tretinoin.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain and Vitaxin;

The subject invention also includes isotopically-labelled compounds, which are identical to those recited in Formula I or II but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O ¹⁷O, ³¹P, ³²P, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I or II of this invention and prodrugs thereof can generally be prepared by substituting a readily available isotopically labeled reagent for a non-isotopically labelled reagent.

The compounds of Formula I or II and their pharmaceutically acceptable salts and solvates can each independently also furthermore be used in a palliative neo-adjuvant/adjuvant therapy in alleviating the symptoms associated with the diseases recited herein as well as the symptoms associated with abnormal cell growth. Such therapy can be a mono-therapy or can be in a combination with chemotherapy and/or immunotherapy.

The terms “abnormal cell growth” and “hyperproliferative disorder” are used interchangeably in this application.

“Abnormal cell growth”, as used herein, refers to cell growth that is independent of normal regulatory mechanisms (e.g., loss of contact inhibition), including the abnormal growth of normal cells and the growth of abnormal cells. This includes, but is not limited to, the abnormal growth of: (1) tumor cells (tumors), both benign and malignant, expressing an activated Ras oncogene; (2) tumor cells, both benign and malignant, in which the Ras protein is activated as a result of oncogenic mutation in another gene; (3) benign and malignant cells of other proliferative diseases in which aberrant Ras activation occurs. Examples of such benign proliferative diseases are psoriasis, benign prostatic hypertrophy, human papilloma virus (HPV), and restinosis. “Abnormal cell growth” also refers to and includes the abnormal growth of cells, benign and malignant, resulting from activity of the enzyme farnesyl protein transferase.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

A “suitable substituent” is intended to mean a chemically and pharmaceutically acceptable functional group i.e., a moiety that does not negate the inhibitory activity of the inventive compounds. Such suitable substituents may be routinely selected by those skilled in the art. Illustrative examples of suitable substituents include, but are not limited to halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O) groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, arylsulfonyl groups and the like.

As used herein, the term “alkyl,” as well as the alkyl moieties of other groups referred to herein (e.g., alkoxy), may be linear or branched (such as methyl, ethyl, n-propyl, isopropyl, n-butyl, iso-butyl, secondary-butyl, tertiary-butyl), and they may also be cyclic (e.g., cyclopropyl or cyclobutyl); optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said alkyl” as used herein refers to any of the preceding alkyl moieties within a group such as alkoxy, alkenyl or alkylamino. Preferred alkyls include (C₁-C₄) alkyl, most preferably methyl.

As used herein, the term “cycloalkyl” refers to a mono or bicyclic carbocyclic ring (e.g., cyclopropyl, cyclobutyl, cyclopentyl, cyclohexyl, cycloheptyl, cyclooctyl, cyclononyl, cyclopentenyl, cyclohexenyl, bicyclo[2.2.1] heptanyl, bicyclo[3.2.1]octanyl and bicyclo[5.2.0]nonanyl, etc.); optionally containing 1-2 double bonds and optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said alkyl” as used herein refers to any of the preceding alkyl moieties within a group such alkoxy, alkenyl or alkylamino. Preferred cycloalkyls include cyclobutyl, cyclopentyl and cyclohexyl.

As used herein, the term “halogen” includes fluoro, chloro, bromo or iodo or fluoride, chloride, bromide or iodide.

As used herein, the term “alkenyl” means straight or branched chain unsaturated radicals of 2 to 6 carbon atoms, including, but not limited to ethenyl, 1-propenyl, 2-propenyl (allyl), iso-propenyl, 2-methyl-1-propenyl, 1-butenyl, 2-butenyl, and the like; optionally substituted by 1 to 3 suitable substituents as defined above such as fluoro, chloro, trifluoromethyl, (C₁-C₆) alkoxy, (C₆-C₁₀) aryloxy, trifluoromethoxy, difluoromethoxy or (C₁-C₆)alkyl.

As used herein, the term “(C₂-C₆)alkynyl” is used herein to mean straight or branched hydrocarbon chain radicals having one triple bond including, but not limited to, ethynyl, propynyl, butynyl, and the like; optionally substituted by 1 to 5 suitable substituents as defined above such as fluoro, chloro, trifluoromethyl, (C₁-C₆)alkoxy, (C₆-C₁₀) aryloxy, trifluoromethoxy, difluoromethoxy or (C₁-C₆)alkyl.

As used herein, the term “carbonyl” or “(C=0)” (as used in phrases such as alkylcarbonyl, alkyl-(C=0)- or alkoxycarbonyl) refers to the joinder of the >C=0 moiety to a second moiety such as an alkyl or amino group (i.e. an amido group). Alkoxycarbonylamino (i.e. alkoxy(C=0)- NH—) refers to an alkyl carbamate group. The carbonyl group is also equivalently defined herein as (C=0). Alkylcarbonylamino refers to groups such as acetamide.

As used herein, the term “aryl” means aromatic radicals such as phenyl, naphthyl, tetrahydronaphthyl, indanyl and the like; optionally substituted by 1 to 5 suitable substituents as defined above such as fluoro, chloro, trifluoromethyl, (C₁-C₆)alkoxy, (C₆-C₁₀) aryloxy, trifluoromethoxy, difluoromethoxy or (C₁-C₆)alkyl.

As used herein, the term “heteroaryl” refers to an aromatic heterocyclic group usually with one heteroatom selected from O, S and N in the ring. In addition to said heteroatom, the aromatic group may optionally have up to four N atoms in the ring. For example, heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. Particularly preferred heteroaryl groups include oxazolyl, imidazolyl, pyridyl, thienyl, furyl, thiazolyl and pyrazolyl.

The term “heterocycloalkyl” as used herein means a nonaromatic monovalent ring (which can include bicyclo ring systems) having from 4 to 10 members, of which, up to 4 are heteroatoms such as N, O and S for example. The heterocycloalkyl groups of this invention can also include ring systems substituted with one or more oxo moieties. Heterocycloalkyl groups may be unsubstituted or substituted with those substituents enumerated for cycloalkyl. Examples of heterocycloalkyl groups include, but are not limited to, 2-or 3-tetrahydrothieno, 2- or 3-tetrahydrofurano, 1-, 2- or 3-pyrrolidino, 2-, 4-, or 5-thiazolidino, 2-, 4-, or 5-oxazoli [symbol] dino, 2-, 3-, or 4-piperidino, N-morpholinyl, N-thiamor [symbol] ipholinyl, 3-azabicyclo[3.1.0]hexanyl, 3-azabicyclo[4.1.0]heptanyl, 1,4-dioxaspiro[4.5]decyl, 1,4-dioxaspiro[4.4]nonyl, 1,4-dioxaspiro[4.3]octyl, and 1,4-dioxaspiro[4.2]heptyl. Examples of substituted heterocycloalkyl groups include, but are not limited to, 1-methyl-pyrrolidin-3-yl, 1-acetyl-pyrrolidin-3-yl, 1-methyl-piperidin-4-yl, 1-acetylpiperidin-4-yl, 1-methyl-azetidin-3-yl, 1-acetyl-azetidin-3-yl, 2-oxo-piperidin-1-yl, and 2,3-Dimethyl-1,4-dioxa-spiro[4.4]nonyl.

As used herein, the phrase “heterocyclic ring” refers to a nonaromatic ring having from 4 to 8 members, of which at least 1 is a N atom, and up to 4 of which are heteroatoms such as N, O and S for example. The heterocyclic ring may be unsubstituted or substituted on a carbon atom with those substituents enumerated for cycloalkyl. Examples of such heterocyclic rings include pyrrolidine, piperidine, piperazine, morpholine, and thiamorpholine.

The term “alkoxy”, as used herein, unless otherwise indicated, means O-alkyl groups wherein “alkyl” is as defined above.

The term “acyl”, as used herein, refers to a species containing a carbon-oxygen double bond.

The phrase “pharmaceutically acceptable salt(s)”, as used herein, unless otherwise indicated, includes salts of acidic or basic groups which may be present in the compounds of formula I or II. The compounds of formula I or II that are basic in nature are capable of forming a wide variety of salts with various inorganic and organic acids. The acids that may be used to prepare pharmaceutically acceptable acid addition salts of such basic compounds of Formula I or II are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphtho ate)] salts.

Those compounds of the Formula I or II that are acidic in nature are capable of forming base salts with various pharmacologically acceptable cations. Examples of such salts include the alkali metal or alkaline earth metal salts and particularly, the sodium and potassium salts.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula I and II may also exist as tautomers. This invention relates to the use of all such tautomers and mixtures thereof.

This invention also encompasses pharmaceutical compositions containing and methods of treating proliferative disorders or abnormal cell growth through administering prodrugs of compounds of the formula I and II. Compounds of formula I and II having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula I and II. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Each of the patents, patent applications, published International applications, and scientific publications referred to in this patent application is incorporated herein by reference in its entirety.

The compounds of the present invention are readily prepared according to synthetic methods familiar to those skilled in the art. The compounds of formula I or II that are basic in nature are capable of forming a wide variety of different salts with various inorganic and organic acids. Although such salts must be pharmaceutically acceptable for administration to animals, it is often desirable in practice to initially isolate the compound of formula I or II from the reaction mixture as a pharmaceutically unacceptable salt and then simply convert the later back to the free base compound by treatment with an alkaline reagent and subsequently convert the latter free base to a pharmaceutically acceptable acid addition salt. The acid addition salt of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula I and II that are acidic in nature are capable of forming base salts with various pharmacologically acceptable cations. Examples of such salts include the alkali metal or alkaline-earth metal salts and particularly, the sodium and potassium salts.

These salts are all prepared by conventional techniques. The chemical bases which are used as reagents to prepare the pharmaceutically acceptable base salts of this invention are those, which form non-toxic, base salts with the acidic compounds of formula I or II. Such non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intraperitoneal, intravascular or infusion), topical, and rectal administration.

The amount of the active compound administered will be dependent on the subject being treated, the severity of the disorder or condition, the rate of administration and the judgment of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumor substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-(N-(3,4-dihy-dro-2-methyl-4-oxoquinazolin-6-ylmethyl)-N-methy lamino]-2-thenoyl)-L-glutamic acid; growth factor inhibitor; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-trifluoromethyl) propionanilide). Such conjoint treatment may be achieved by way of simultaneous, sequential or separate dosing of the individual components of the treatment.

The pharmaceutical composition may, for example, be in a form suitable for oral administration as a tablet, capsule, pill, powder, sustained release formulations, solution, and suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For example, see Remington's Pharmaceutical Sciences, Mack Publishing Company, Easter, Pa., 15th Edition (1975).

### Example I

### Isolation of Compound A (Kuminol) and Compound B (Vernoginin)

Vernonia guineensis Benth. leaf material was harvested and dried under shade. Following drying, the leaf material was ground into powder. One thousand five hundred grams of the powder was extracted twice with an adequate volume of acetone solvent at a temperature of 20-25° C. for 48 hours. The extract was filtered using a Buckner funnel with 0.5 micron filter paper to separate the liquid extract from the marc. The liquid extract was subjected to drying in-vacuo using a rotavapor to collect a dark gummy residue.

The anticancer activity of the extract obtained as described above was confirmed using the highly proliferative B16 cell line. The bioactive gummy residue was then fractionated by absorbing onto Celite, drying and extracting with increasing polarities of ethyl acetate in hexane. Each of the fractions obtained was evaporated in-vacuo to remove the solvent and monitored by thin layer chromatography (TLC). Fractions that showed similarities were pooled. The dried residue of each fraction was used in the anticancer assay to determine effect on cell proliferation. Fraction #4 extracted with hexane:ethyl acetate (1:1) exhibited activity in anticancer assay. The active fraction was subjected to dry column chromatography (DCC) using normal phase silica gel activated for visualization under UV 254 detector. A 38 mm×750 mm DCC was loaded with 250 grams of normal phase silica gel. The sample was adsorbed onto 20 g silica and dried before loading onto the top of the packed DCC. The DCC was developed using ethyl acetate:hexane (6:4). The developed column revealed three UV active bands which were carefully cut into sections using a sharp knife. The UV active bands and other non active sections were extracted with ethyl acetate and ran on TLC. A total of 11 fractions were obtained after TLC evaluation and pooling. Fractions 6 and 7 demonstrated anticancer activity in the anticancer assay.

Compound A (Kuminol) was obtained as white powder from fraction 7 after concentration of the ethyl acetate extract. The analysis of this compound showed the molecular formula of C₁₉H₂₄O₈ with a molecular weight of 380.39. NMR analysis showed the ¹H-NMR (DMSO-d₆, 400 MHz instrument), δ (ppm)=1.72, 1.81, 1.98, 2.19, 2.42, 2.51, 3.32, 4.10, 5.10, 5.12, 5.33, 5.82, 5.86, 6.21, 6.55, 6.58 and 9.46; ¹³C-NMR (DMSO-d₆, 400 MHz instrument) 6 (ppm)=16.55, 22.06, 24.29, 45.40, 48.48, 59.35, 69.14, 75.76, 124.07, 127.62, 127.84, 131.90, 133.91, 140.61, 141.35, 148.18, 164.52, 168.73 and 195.26.

Fraction 6 of the DCC was rechromatographed using a Waters Sep Pack flash chromatography system with normal phase silica gel cartridge to obtain Compound B (Vernoginin) as a colorless oily substance with molecular formula of C₂₁H₃₀O₇ and a molecular weight of 394.46. The spectroscopic analysis of this compound showed that ¹H -NMR (DMSO-d₆, 400 MHz instrument) δ (ppm)=1.02, 1.78, 1.82, 1.84, 2.08, 3.18, 3.24, 4.15, 4.83, 4.86, 5.53, 5.54, 5.89, 5.98, 6.15, 6.49 and 9.47; ¹³C-NMR (DMSO-d₆, 400 MHz instrument) δ (ppm)=17.48, 30.64, 41.47, 43.82, 44.10, 50.69, 59.57, 69.06, 77.00, 112.07, 118.93, 124.32, 137.23, 138.92, 140.62, 144.73, 145.83, 164.84, 168.98 and 194.63.

The derived spectra data was used to elucidate the structure of the compounds. The structures of Compounds A and B were submitted to chemical databases for comparison with existing structures. No molecules with similar structures were found.

### Example 2

### Ex Vivo Proliferation Assay for Cancer Cells

### Cell Proliferation Method:

Breast cancer cells lines MDA-MD-231 and MCF-7, colon cancer cell line HCT-116, Leukaemia cell line HL-60, lung cancer cell line A549, melanoma cancer cell line A375, ovarian cancer cell line OVCAR3 and prostate cancer cell line PC-3 were treated with Compound A (Kuminol) and Compound B (Vernoginin) to determine the effects of the molecules on the proliferation of each cell line.

Established protocol was used to assay the anti-proliferation effects of these compounds against selected cancer cell lines. Each cell line is grown in RPMI/10% FBS/1% glutamine. On the day the experiment is initiated, cells are trypsinized and plated into 96 well plates in 50 ul of media. Compound is added approximately 18 hours after plating. Cells are plated at a density so that 72 hours post drug addition, the cells are in log phase (500-2000 cells/well). The compounds are solubilized in DMSO at a concentration of 100 mM, aliquoted and stored at −20^{C}. On the day of drug addition, the compounds are removed from the freezer and serially diluted in DMSO to concentrations as low as 100 nM. The compounds are then diluted 1:1000 into growth media (this is 2× the final concentration) including a DMSO alone control. 50 ul of media/compound is added to each well. The cells are allowed to proliferate for 72 hours. The experiment is terminated using WST-1 (Roche) and absorbance is read at 450 nm/690 nm IC₅₀s are calculated in GraphPad Prism converting all values to percentage of control cell growth. Each cell line is run in duplicate and the average of the two runs is the calculated IC₅₀ of each compound. The IC₅₀ results of the anti-proliferation effects against the above referenced cancer cell lines are indicated in Table 1.

TABLE 1
Results of cell proliferation assay (IC₅₀ in uM)

| | Kuminol | Vernoginin |
|---|---|---|
| Cancer Cell Line | (Compound A) | (Compound B) |
| Breast—MDA-MB-231 | 1.01 ± 0.20 | 0.367 ± 0.03 |
| Breast—MCF-7 | 0.67 ± 0.32 | 1.56 ± 0.96 |
| Colon—HCT-116 | 0.45 ± 0.007 | 0.14 ± 0.15 |
| Leukaemia—HL-60 | 1.55 ± 0.47 | 1.12 ± 0.75 |
| Lung—A549 | 2.04 ± 1.04 | 1.13 ± 0.38 |
| Human melanoma—A375 | 0.35 ± 0.04 | 0.13 ± 0.15 |
| Ovarian—OVCAR3 | 0.74 ± 0.31 | 0.48 ± 0.008 |
| Prostate—PC-3 | 1.00 ± 0.26 | 0.43 ± 0.0.06 |

### Example 3

### Prohibition of Kaposi Sarcoma and Non-Hodgkin's Lymphoma Cell Proliferation

Kaposi sarcoma (KSY-1) and non-Hodgkin's lymphoma (NHBL) cells were treated with Kuminol (Compound A) and Vernoginin (Compound B) to determine the effects of the molecules on the proliferation of each cell line.

KSY-1 and NHBL cells were cultured in 75 cm² flasks in 20 ml of RPMI 1640 with 10% fetal bovine serum (FBS) at 37° C. in 5% CO₂ in air. Subconfluent monolayers between passages 19 and 25 were used for the experiments.

KSY-1 and NHBL cells were seeded in 24-well plates (10⁵ cells/well) and allowed to attach for 24 h. Cells were then exposed to varied concentrations of Kuminol (Compound A) and Vernoginin (Compound B) and then incubated at 37° C. for 48 h in 5% CO₂ in air. The drugs were dissolved in DMSO and the final DMSO concentration in the media was 0.1%. The Trypan Blue assay was used to determine effect of Kuminol and Vernoginin on each cell line. Following the manufacturer's protocol, at the end of 48 hrs of incubation, cell dissociation buffer was added to each well and left for 5 minutes. 1 ml of the cells were transferred into a 15 ml tube and 10 ml PBS added to it. The cells were centrifuged at 1600 rpm for 10 min. The cells were resuspended in 1 ml RPMI media. The media containing cells was mixed 1:1 with Trypan Blue (0.4%), Sigma and live cells illuminated by the Trypan Blue were counted under microscope.

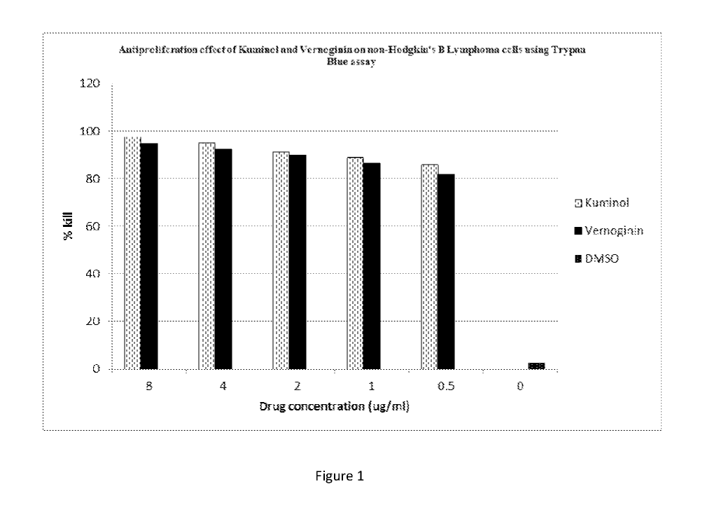

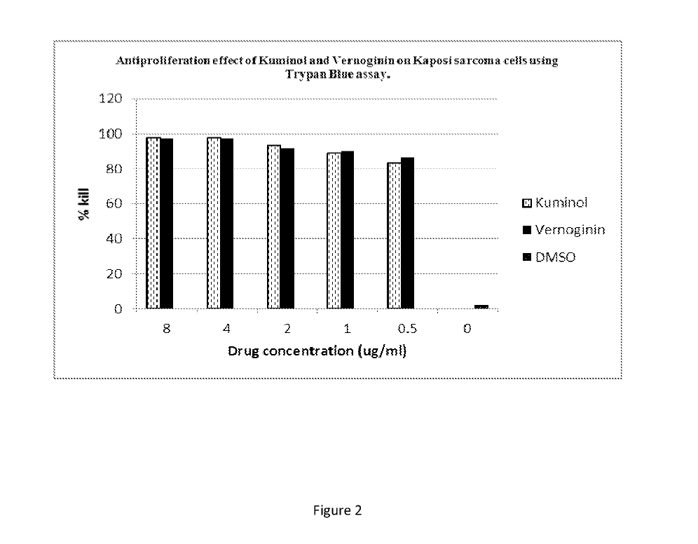

Results of the cell proliferation effect of Kuminol and Vernoginin on KSY-1 and NHBL are shown in FIGS. 1 and 2.

### Example 4

### Apoptosis Determination

The apoptotic effects of Kuminol (Compound A) and Vernoginin (Compound B) against the human leukaemia cell line HL-60 was determined. Annexin V and Propidium Iodide assay were carried out to determine whether Kuminol and Vernoginin have effect on cell cycle and induction of apoptosis in cancer cells. Cell death can result from either necrosis or apoptosis (programmed cell death). Necrotic cell death produces a negative effect as the lysed cells generally lead to an inflammatory response whereas death by apoptosis do not produce any inflammatory response as the lysed cells are eliminated by phargocytosis.

The human leukaemia cell line HL60 were plated in RPMI1640 at 4×10⁶ (2×10⁵ cells/ml) and treated with 1×IC₅₀, 10×IC₅₀ and 50×IC₅₀ Kuminol and Vernoginin. For cell cycle, 2 ml of the treated cells were stained with propidium iodide as per the manufactures instruction, aliquots (2 ml) of cells were removed and fixed at 3, 6, 24, 48 and 72 hours. For apoptosis, 2 ml aliquots of cells were removed at 0, 3, 6 and 24 hours and were stained with Annexin V FITC as per the manufacturer's instructions. The presence of apoptosis and cell cycle changes were detected using a Cytomics 500 Beckman Coulter Flow Cytometer (Coulter, Inc. Hialeah, Fla., USA

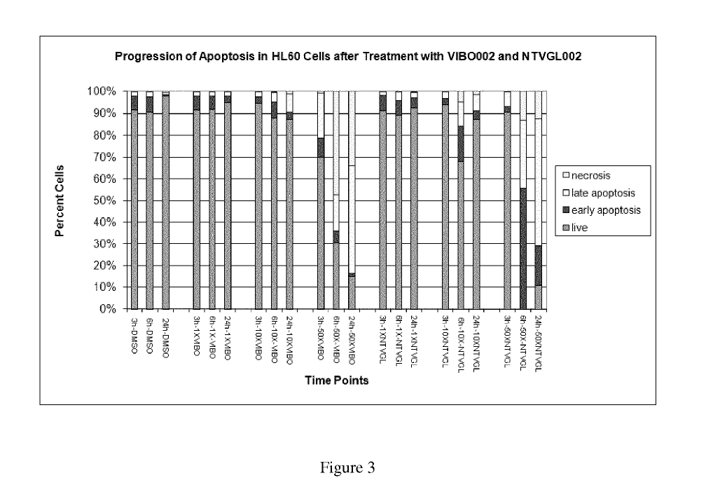

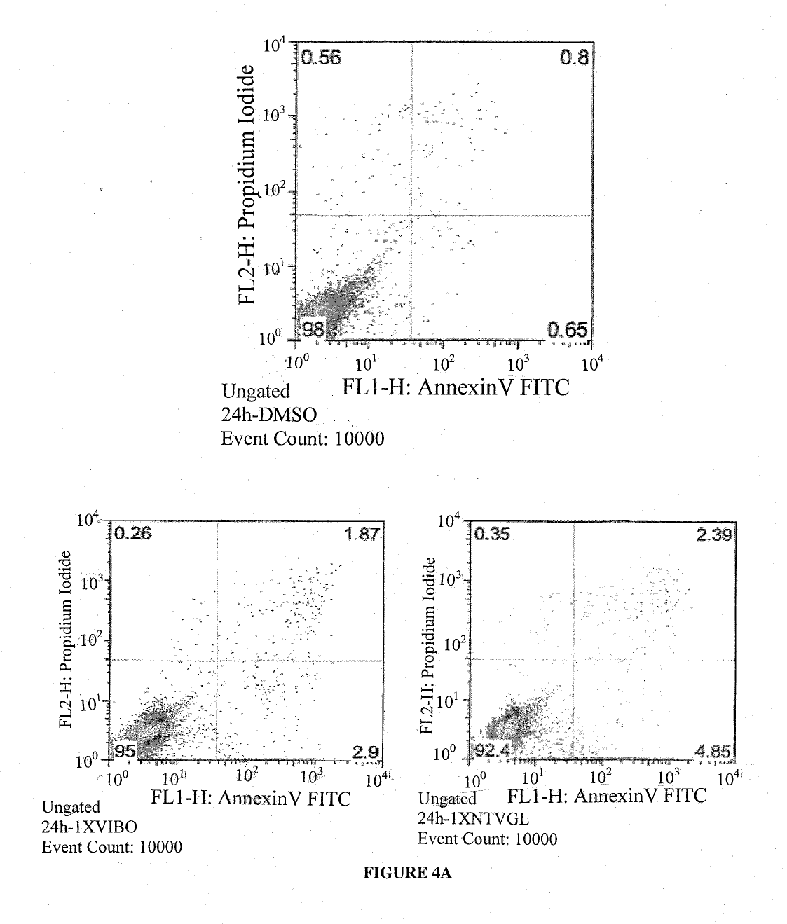

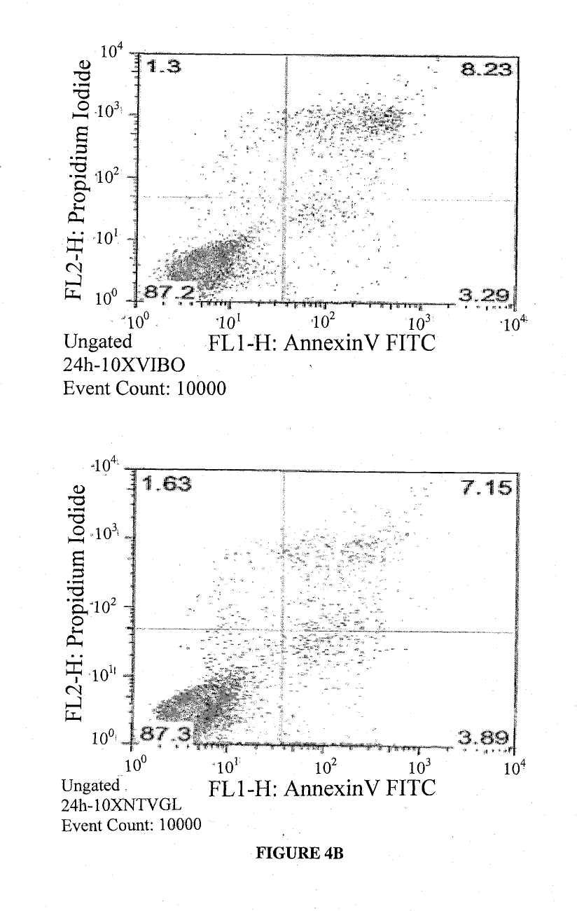

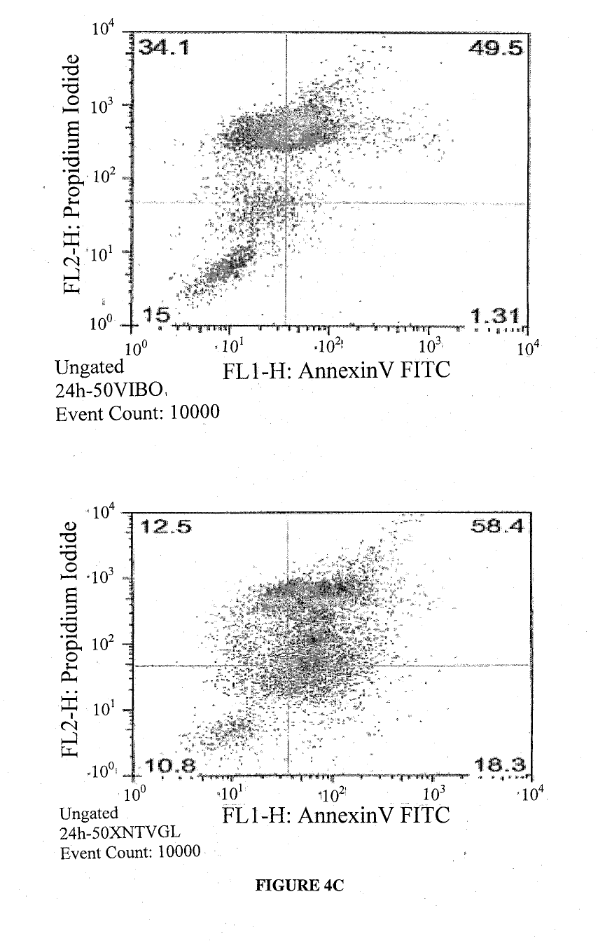

The results from this assay demonstrate that Kuminol (Compound A) and Vernoginin (Compound B) induce apoptosis in this cell line (FIGS. 3 & 4).

### Example 5

### Proof of In Vivo Antitumor Activity

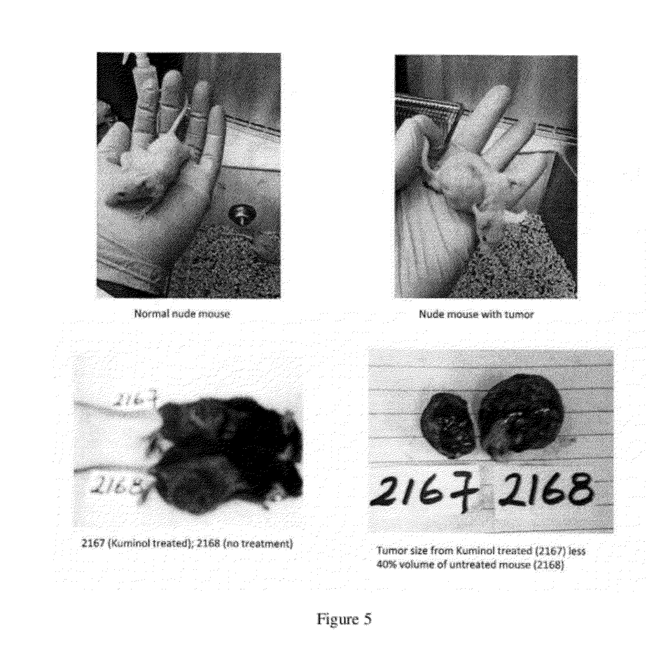

FIG. 5 illustrates the suppression effects of compound A (Kuminol) on B16 tumors in C57 mice. Histological examination of tumors from the treated and untreated mice reveals that the drug might be acting via an antiangionesis mechanism.

### REFERENCES

Alembert T. Tchinda, Apollinaire Tsopmo, Pierre Tane, Johnson F. Ayafor, Joseph D. Connolly, Olov Sterner, 2002. Vernoguinosterol and vernoguinoside, trypanocidal stigmastane derivatives from Vernonia guineensis (Asteraceae). Phytochemistry 59: 371-374.
Alembert T. Tchinda, Apollinaire Tsopmo, Pierre Tane, Johnson F. Ayafor, Joseph D. Connolly, 2003. Stigmatane derivatives and isovaleryl sucrose esters from Vernonia guineensis (Asteraceace). Phytochemistry 63 (7): 841-846.
Burkill H. M., 1985. The Useful plants of West Tropical Africa. Ed 2, Vol 1. Royal Botanic Gardens Kew. UK. P.510
Igile, G., Oleszek, W., Jurzysta, M., Aquino, R., de Tommasi, N., Pizza, C., 1995. Venoniosides D and E, two novel saponins from Vernonia amygdalina. J. Nat. Prod. 58, 1438-1443.
Iwu, M. M., 1993. Handbook of African Medicinal Plants. CRC Press, London. 415p. Jisaka, M., Ohigashi, H., Takagaki, T., Nozaki, H., Tada, T., Hirota, M., Irie, R., Huffman, M. A., Nishida, T., Kaji, M., Koshimizu, K., 1992. Bitter steroid glucosides, vernoniosides A1, A2 and A3, and related B1 from a possible medicinal plant, Vernonia amygdalina, used by wild chimpanzees. Tetrahedron 48, 625-632.
Jisaka, M., Ohigashi, H., Kazunori, T., Hirota, M., Irie, R., Huffman, M. A., Koshimizu, K., 1993. Steroid glycosides from Vernonia amygdalina, a possible chimpanzee medicinal plant. Phytochemistry 34, 409-413.
Noumi Emmanuel, 2010. Ethno medicines used for treatment of prostatic disease in Foumban, Cameroon. African J. of Pharmacy and Pharmacology, Vol 4(11):793-805.
Ponglux, D., Wongseripipatana, S., Aimi, N., Oya, N., Hosokawa, H., Haginiwa, J., Sakai, S., 1992. Structures of two new bitter principles isolated from a That medicinal plant Vernonia extensa. Chem. Pharm. Bull. 40, 553-555.
Raoul Toubiana, Bernard Mompon, Chi Man Ho, Marie-Joséphe Toubiana, 1975. Isolement du vernodalin et du vernolepin a partir de Vernonia guineensis: AUthenticité du squelette elemane. Phytochemistry Vol 14(3):775-778.
Sanogo, R., Germano, M. P., de Tommasi, N., Pizza, C., Aquino, R., 1998. Vernoniosides and an androstane glycoside from Vernonia kotschyana. Phytochemistry 47, 73-78.

1. A compound having the following Formula I: 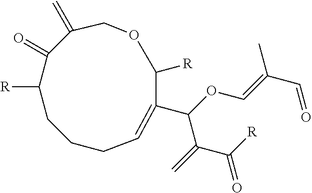
wherein “R” is independently methyl(CH₃), hydroxyl (—OH), methoxy (—OCH₃), ester (—OCH₂CH₃, —OCCH₂CH₂COOH), or (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₁₈)cyclo alkyl, (C₆-C₁₀) aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula I; or a prodrug or pharmaceutically active metabolite of a compound of the Formula I, or a pharmaceutically acceptable salt of a prodrug or metabolite thereof.

2. The Compound of claim 1, wherein the compound is Kuminol and R is a hydroxyl group.

3. A compound having the following Formula II: 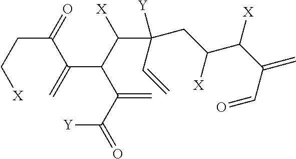
wherein “X” and “Y” are independently methyl (CH₃), hydroxyl (—OH), methoxy (—OCH₃), ester (—OCH₂CH₃, —OCCH₂CH₂COOH) or (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy., or a pharmaceutically acceptable salt of a compound of the Formula I; or a prodrug or pharmaceutically active metabolite of a compound of the Formula I, or a pharmaceutically acceptable salt of a prodrug or metabolite thereof.

4. The compound of claim 3, wherein the compound is Vernoginin and X is a hydroxyl group and Y a methyl group.

5. The compounds as claimed in claims 1 and 3, wherein the compounds are isolated from Vernonia guineensis Benth.

6. A method for treating abnormal cell growth comprising the step of administering to a patient in need of such treatment, a pharmaceutical composition comprising organic solvent extract from the leaves of Vernonia guineensis Benth.

7. A pharmaceutical composition comprising an effective amount for treating cancer of an agent selected from: a compound of the Formula I, wherein R is independently methyl, or hydroxyl, or (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₁₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula I; or a prodrug or pharmaceutically active metabolite of a compound of the Formula I, or a pharmaceutically acceptable salt of a prodrug or metabolite thereof.

8. A pharmaceutical composition comprising an effective amount for treating cancer of an agent selected from: a compound of the Formula II, wherein X and Y are independently methyl, or hydroxyl, or selected from (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula II; or a prodrug or pharmaceutically active metabolite of a compound of the Formula II, or a pharmaceutically acceptable salt of a prodrug or metabolite thereof.

9. A method of treating cancer comprising administering to a patient in need of such treatment, a pharmaceutical composition comprising an effective amount for treating cancer selected from: a compound of the Formula I, wherein R is independently methyl, or hydroxyl, or (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula I; or a prodrug or pharmaceutically active metabolite of a compound of the Formula I, or a pharmaceutically acceptable salt of a prodrug or metabolite thereof.

10. A method of treating cancer comprising administering to a patient in need of such treatment, a pharmaceutical composition comprising an effective amount for treating cancer selected from: a compound of the Formula II, wherein X and Y are independently methyl, or hydroxyl, (C₁-C₆)alkylC(O), (C₁-C₆)alkyl, (C₃-C₈)cycloalkyl, (C₆-C₁₀)aryl, (C₁-C₁₀)heteroaryl, (C₁-C₁₀)heterocycloalkyl; wherein each of the aforesaid (C₁-C₆)alkylC(O), (C₁-C₆)alkyl, (C₃-C₈)cycloalkyl, (C₆-C₁₀)aryl, (C₁-C₁₀)heterocycloalkyl, and (C₁-C₁₀)heteroaryl groups is independently optionally substituted with 1 to 5 substituents independently selected from halogen, (C₁-C₆)alkyl and (C₁-C₆)alkoxy, or a pharmaceutically acceptable salt of a compound of the Formula II; or a prodrug or pharmaceutically active metabolite of a compound of the Formula II, or a pharmaceutically acceptable salt of a prodrug or metabolite thereof.

11. The method of claim 8 or 9 wherein the cancer cells are particularly prostate cancer, breast cancer, human melanoma, lung cancer, ovarian cancer, leukaemia, colon cancer, Kaposi sarcoma and Non-Hodgkin's B Lymphoma.

12. A method for the treatment of abnormal cell growth in a mammal comprising administering to said mammal an amount of a compound of claim 1 or 3 or a pharmaceutically acceptable salt, prodrug, solvate or hydrate thereof that is effective in treating said abnormal cell growth.

13. A method for the treatment of vasculogenesis, restenosis, atherosclerosis or angiogenesis in a mammal comprising administering to said mammal a therapeutically effective amount of a compound of claim 1 or 3 or a pharmaceutically acceptable salt, prodrug, solvate or hydrate thereof that is effective in treating said vasculogenesis, restenosis, atherosclerosis or angiogenesis.

14. A method for the treatment of a hyperproliferative disorder in a mammal which comprises administering to said mammal a therapeutically effective amount of a compound of claim 1 or 3 or a pharmaceutically acceptable salt, prodrug, solvate or hydrate thereof in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.
